(19) United States
(12) Patent Application Publication
(10) Pub. No.: US 2021/0220392 A1
(43) Pub. Date: Jul. 22, 2021

(54) MOLECULAR HYDROGEN-CONTAINING COMPOSITION FOR PREVENTION AND/OR IMPROVEMENT OF INFLAMMATORY BOWEL DISEASE

(71) Applicant: MiZ Company Limited, Kamakura-shi (JP)

(72) Inventors: Fumitake SATO, Kanagawa (JP); Yusuke ICHIKAWA, Kanagawa (JP); Shinichi HIRANO, Kanagawa (JP); Ryosuke KUROKAWA, Kanagawa (JP); Masatsugu SAITO, Kanagawa (JP)

(21) Appl. No.: 17/151,849

(22) Filed: Jan. 19, 2021

(30) Foreign Application Priority Data
Jan. 21, 2020 (JP) 2020-019226

(51) Int. Cl. A61K 33/00

(52) U.S. Cl. CPC A61K 33/00

(57) ABSTRACT

The present invention provides a composition for prevention and/or improvement of inflammatory bowel disease. More specifically, the present invention provides a composition for prevention and/or improvement of inflammatory bowel disease and a symptom associated with inflammatory bowel disease in a subject, comprising molecular hydrogen as an active ingredient.

### RELATED APPLICATIONS

This application claims priority to Japanese Patent Application No. 2020-019226, filed on Jan. 21, 2020, the entire content of which is incorporated herein by reference.

### BACKGROUND OF THE INVENTION

### 1. Field of the Invention

The present invention provides a molecular hydrogen-containing composition for prevention and/or improvement of inflammatory bowel disease in subjects.

### 2. Description of the Related Art

Inflammatory bowel disease is a disease in which a body's own immune cells attack intestinal cells because of abnormality in the human immune system, leading to inflammation of the intestines. Patients have symptoms including chronic diarrhea, hematochezia, and abdominal pain. Inflammatory bowel disease is a term mainly used to describe two diseases: ulcerative colitis and Crohn's disease. Both of these diseases are likely to develop in relatively younger populations, and the prevalence in Japan is increasing every year. Inflammatory bowel disease also includes a relatively rare disease called intestinal Bechet's disease. Usually, this disease is not life-threatening, but is rarely cured completely once it develops, requiring lifetime continuation of treatment.

It has been pointed out that hydrogen, the active ingredient of the present invention, has anti-oxidative reactivity, which reduces oxidative stress caused by reactive oxygen species, treats bacterial translocation, and can improve enteric bacteria flora (Japanese Patent No. 6601851). However, there are no cases of refractory inflammatory bowel disease for which clinical effectiveness of hydrogen has been documented.

Prevention and/or improvement of inflammatory bowel disease would make it possible not only to achieve relief of distress and improvement in quality of life for patients, but also to contribute to reduction in healthcare costs. As described above, few components or substances are known to be useful for prevention and/or improvement of inflammatory bowel disease.

Under such circumstances, an object of the present invention is to prevent and/or improve inflammatory bowel disease by using molecular hydrogen and/or to promote improvement of a symptom associated with inflammatory bowel disease.

### SUMMARY OF THE INVENTION

That is, the present invention encompasses the following characteristics:

(1) A composition for prevention and/or improvement of inflammatory bowel disease and a symptom associated with inflammatory bowel disease in a subject, comprising molecular hydrogen as an active ingredient.

(2) The composition according to (1), wherein the inflammatory bowel disease is selected from the group consisting of ulcerative colitis, Crohn's disease, and intestinal Bechet's disease.

(3) The composition according to (1) or (2), wherein the symptom associated with inflammatory bowel disease is selected from the group consisting of chronic diarrhea, hematochezia, abdominal pain, pyrexia, anemia, hemorrhoids, weight loss, polyp development, and psychiatric diseases associated with these symptoms, such as a depressive symptom and insomnia.

(4) The composition according to any of (1) to (3), which is a liquid or a gas comprising the molecular hydrogen.

(5) The composition according to (4), wherein the liquid comprising the molecular hydrogen has a hydrogen concentration of 1 to 10 ppm.

(6) The composition according to (4), wherein the gas comprising the molecular hydrogen has a hydrogen concentration of higher than zero (0) and not higher than 18.5% by volume.

(7) The composition according to any of (1) to (6), wherein the subject is a mammalian including a human.

(8) The composition according to any of (1) to (7), which is produced by using a hydrogen gas generating apparatus, a hydrogen water generating apparatus, or a hydrogen gas adding apparatus.

### DETAILED DESCRIPTION OF THE PREFERRED EMBODIMENTS

The present invention can prevent and/or improve inflammatory bowel disease in subjects.

The present invention will be described in more detail below.

### 1. Composition for Prevention and/or Improvement of Inflammatory Bowel Disease

The present invention provides a composition comprising molecular hydrogen as an active ingredient which promotes recovery from or improvement of surgical invasion and/or symptoms associated with surgery in subjects undergoing the surgery.

In the present invention, the term “intestines” refers to the digestive organ involved in food digestion, which begins at the oral cavity, continues through the pharynx, esophagus, stomach, small intestines (duodenum, jejunum, and ileum), and large intestines, and ends at the anus.

In the present specification, the term “inflammatory bowel disease” refers to a disease in which the body's own immune cells attack intestinal cells because of abnormality in the human immune system, leading to inflammation of the intestines. The inflammatory bowel disease is a disease selected from the group consisting of ulcerative colitis, Crohn's disease, and intestinal Bechet's disease.

In the present specification, the term “symptom associated with inflammatory bowel disease” refers to a symptom that accompanies inflammatory bowel disease. Examples of such a symptom include chronic diarrhea, hematochezia, abdominal pain, pyrexia, anemia, hemorrhoids, weight loss, polyp development, as well as psychiatric diseases associated with these symptoms, such as a depressive symptom and insomnia.

In the present specification, the term “inflammatory colitis” refers to Intractable Disease 97, which is designated by the Ministry of Health, Labour and Welfare of Japan as of January 2020.

In the present specification, the term “Crohn's disease” refers to Intractable Disease 96, which is designated by the Ministry of Health, Labour and Welfare of Japan as of January 2020.

In the present specification, the term “intestinal Bechet's disease” refers to Intractable Disease 56, which is designated by the Ministry of Health, Labour and Welfare of Japan as of January 2020.

In the present specification, the term “subject” includes mammalians such as primates including humans, pet animals such as dogs and cats, and ornamental animals such as zoo animals. Preferred subjects are humans.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen (i.e., the composition of the present invention) is higher than zero (0) and not higher than 18.5% by volume, for example, 0.5% to 18.5% by volume, preferably 1% to 10% by volume, for example, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, and the like. In the present invention, higher hydrogen gas concentrations (but below the explosion limit) or higher daily hydrogen doses tend to be associated with greater effects of promoting prevention and/or improvement (e.g., suppression or alleviation) of inflammatory bowel disease.

Because hydrogen is a flammable and explosive gas, it is preferable to add hydrogen to the composition of the present invention under conditions safe for subjects such as humans and administer the mixture to subjects to prevent and/or improve inflammatory bowel disease.

When a gas other than hydrogen gas is air, the air concentration is in the range of, for example, 81.5% to 99.5% by volume.

When a gas other than hydrogen gas is a gas containing oxygen gas, the oxygen gas concentration is in the rage of, for example, 21% to 99.5% by volume.

As another main gas, for example, nitrogen gas can be further added.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

In the present invention, higher concentrations of dissolved hydrogen (but below the explosion limit) or higher daily hydrogen doses tend to be associated with greater effects of preventing and/or improving inflammatory bowel disease.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

A gas containing molecular hydrogen or a liquid containing molecular hydrogen (e.g., water [e.g., purified water, sterilized water], physiological saline, drip infusion solution) prepared using the above-mentioned apparatuses or devices can be administered orally or parenterally to subjects with inflammatory bowel disease preoperatively, perioperatively, or postoperatively.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas is inhaled from the mouth or the nose via a nasal cannula or a mask-like device covering the mouth and the nose, transported to the lungs, and delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

### 2. A Method for Preventing and/or Improving Inflammatory Bowel Disease

The present invention further provides a method for promoting recovery from surgical invasion and/or recovery from or improvement of symptoms associated with surgery in subjects with inflammatory bowel disease, using the above-mentioned composition comprising molecular hydrogen as an active ingredient.

The composition containing molecular hydrogen, inflammatory bowel disease, symptom associated with inflammatory bowel disease, dose, administration method, and the like are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Or, in the method of the present invention, for example, 200 to 500 mL per dose for intravenous administration or, for example, 500 to 1000 mL per dose for oral administration of a liquid containing molecular hydrogen at a concentration of, for example, 1 to 10 ppm, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, or the like, preferably 3 to 10 ppm, 4 to 10 ppm, 5 to 10 ppm, 5 to 9 ppm, 5 to 8 ppm, 5 to 7 ppm, or the like can continue to be administered to subjects for, for example, 0.5 to three months or longer, four to seven months or longer, one to three years or longer.

The method of the present invention may further be used in combination with a therapeutic agent used for the treatment of inflammatory bowel disease, if necessary. Such a combination use is expected to increase levels of prevention and/or improvement of inflammatory bowel disease.

### EXAMPLE

The present invention is explained more specifically with reference to the following example. However, the example is not intended to limit the scope of the present invention.

### Example 1

### A Case of Ulcerative Colitis Improved by Hydrogen Inhalation

A 60-year-old male patient who was diagnosed as having ulcerative colitis had a symptom of melena during defecation with a 50% probability, which caused anemia. From around January 2018, the patient was taking a therapeutic agent for ulcerative colitis but was distressed because the disease was not cured with cyclic improvement and worsening. Additionally, the patient became depressed and gloomy, suffering insomnia and weight loss, because of the prolonged symptoms of ulcerative colitis.

An endoscopy revealed that the rectum (i.e., between the large intestine and the anus) was filled with blood, and the wall of the small and large intestines roughened.

On Jun. 3, 2019, the patient initiated inhalation of hydrogen gas. The hydrogen gas was inhaled for one to three hours per day using a hydrogen generating machine (type, Jobs-α; a hydrogen concentration, approximately 5.0%; generation rate of hydrogen gas, approximately 200 mL/min) manufactured by MiZ Company Limited.

The patient inhaled hydrogen gas and drank hydrogen water (7 WATER at a concentration of 7 ppm and filling a 500 mL PET bottle manufactured by MiZ Company Limited). The patient drank two 500 mL PET bottles of hydrogen water per day.

The patient continued inhalation of hydrogen gas virtually every day until August 22, when the inhalation therapy was completed, although he missed inhalation on one or two days.

The volume of melena during defecation began to decrease around June 9, six days after the initiation of hydrogen gas inhalation. Additionally, the patient's sleep condition also improved. The patient regained his vigor and felt younger, and his general condition recovered.

By Jun. 17, 2019, hemorrhage during defecation had further decreased. The stool that had been watery became solid and recovered to an extent that only a small amount of blood was attached to the stool. The frequency of defecation and the patient's vigor returned to the levels before the onset of the disease.

By Jul. 31, 2019, no hemorrhage was found except that blood was attached to the stool only on the day after the patient had drunk alcohol.

By Aug. 22, 2019, no hemorrhage was found, and the patient's body weight increased to the healthy weight before the onset of the disease.

On Aug. 22, the patient discontinued the inhalation of hydrogen gas and only continued to drink hydrogen water thereafter. His condition was favorable, and ulcerative colitis did not recur.

The endoscopy performed on Sep. 25, 2019 revealed that there was no hemorrhage in the rectum, which had been filled with blood, and the wall of the small and large intestines recovered completely. Furthermore, the several polyps found in the stomach had disappeared, demonstrating that polyps had been eliminated by inhalation of hydrogen gas and drinking of hydrogen water.

The present invention can prevent and/or improve inflammatory bowel disease by administering a composition containing molecular hydrogen.

What is claimed is:

1. A method for preventing and/or improving inflammatory bowel disease and a symptom associated with inflammatory bowel disease in a subject, comprising administering an effective amount of molecular hydrogen.

2. The method according to claim 1, wherein the inflammatory bowel disease is selected from the group consisting of ulcerative colitis, Crohn's disease, and intestinal Bechet's disease.

3. The method according to claim 1, wherein the symptom associated with inflammatory bowel disease is selected from the group consisting of chronic diarrhea, hematochezia, abdominal pain, pyrexia, anemia, hemorrhoids, weight loss, polyp development, and psychiatric diseases associated with these symptoms, such as a depressive symptom and insomnia.

4. The method according to claim 1, wherein molecular hydrogen is administered as a liquid composition or a gas composition comprising the molecular hydrogen.

5. The method according to claim 4, wherein the liquid composition has a hydrogen concentration of 1 to 10 ppm.

6. The method according to claim 4, wherein the gas composition has a hydrogen concentration of higher than zero (0) and not higher than 18.5% by volume.

7. The method according to claim 1, wherein the subject is a mammalian including a human.

8. The method according to claim 4, wherein the liquid composition or gas composition is produced by using a hydrogen gas generating apparatus, a hydrogen water generating apparatus, or a hydrogen gas adding apparatus.
